(19) United States
(12) Patent Application Publication
(10) Pub. No.: US 2007/0270786 A1
(43) Pub. Date: Nov. 22, 2007

(54) Acceleration of acrylic bone cement curing time

(75) Inventors: Paul Higham, Ringwood, NJ (US); Robert W. Klein, Chestnut Ridge, NY (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/399,155

(22) Filed: Apr. 6, 2006

(51) Int. Cl. A61B 18/18

(52) U.S. Cl. 606/13

(57) ABSTRACT

A method for accelerating the set time of acrylic bones cements uses ultrasonic energy. Typically acrylic bone cements comprise a mixture of a liquid monomer and a powdered polymer which sets over a period of time to form a hardened material. After the bone cement has been placed in the body the polymerization rate or set time may be increased by the application of ultrasonic sound energy. A horn of an ultrasonic generator is placed in or adjacent to the unset bone cement mixture and acoustic energy is applied which energy accelerates the set time. Typically the frequency is in the range of 20 KHz and an amplitude of between 5 and 30 μm with an application time of between 40 seconds and 2 minutes.

### BACKGROUND OF THE INVENTION

This invention relates to a method for accelerating the curing time for acrylic bone cement. More particularly the invention relates to using ultrasound for curing acrylic bone cement.

Bone cements find wide usage in a variety of applications. For instance, they are used for cementing orthopedic implants in place, for the anchoring of endoprosthesis of the joints, for filling voids in bones, in the treatment of skull defects, and for the performance of spinal fusion. These cements are typically polymeric materials and more particularly acrylic polymers and the surgeon usually mixes the interactive components to make the cement at an appropriate stage during the surgical procedure.

Typically, the components of the bone cement comprise a powdered homopolymer or copolymer of methyl methacrylates, alkyl methacrylates and/or styrene and a suitable liquid monomer. The liquid monomer consists of esters of acrylic or methacrylic acid for example methyl methacrylate. The liquid monomer is typically provided in a glass ampoule. To accelerate the polymerization of the bone cement, a catalyst system may also be used. The catalyst, if present, is in the form of a redox catalyst system, usually containing an organic peroxy compound, such as dibenzoyl peroxide, plus a reducing component, such as p-toluidine. N,N-dimethylparatoluidine (DMPT) can also be used as a polymerization accelerator and hydroquinone (HQ) can be used as a stabilizer. The DMPT and HQ may be included with the liquid monomer. A radiopacifier such as barium sulphate may also be included.

After the bone is prepared the liquid and powered components of the bone cement are mixed. The setting time is one of the most important characteristics of acrylic bone cement. The setting time is the point after mixing at which the cement is hardened enough to bear loading and tested per ASTM 451. Although all bone cement manufacturers indicating the setting profile in their product inserts, the actual setting properties in an operating room (OR) may vary significantly due to different environmental conditions such as temperature, storage conditions, and mixing methods. It is sometimes desirable to accelerate the setting time of a bone cement once the components being cemented are in their proper positions. This allows the surgeon to more quickly complete the implant replacement.

### SUMMARY OF THE INVENTION

It is an aspect of the invention to provide a system for accelerating the curing time of an acrylic bone cement.

It is yet another aspect of the invention to utilize acoustic or sound energy to speed the polymerization of an acrylic bone cement by placing the horn of an ultrasonic sound generator into or adjacent the surface of uncured bone cement.

These and other aspects of the invention are provided by a method for accelerating the set time of acrylic bone cements which cements comprise a mixture of a monomer and a polymer of a two-part acrylic bone cement system. The uncured monomer and polymer mixture is placed in a bone typically surrounding an implant such as an orthopedic implant. After the uncured cement is in place a horn of an ultrasonic sound generator is placed in or adjacent the unset mixture and acoustic or sound energy in the ultrasonic range is applied to the mixture through the horn.

Preferably the sound is in the range of about 20 KHz in frequency and has an amplitude between 5-30 μm (micrometers). The acoustic energy is applied for between 1 and 4 minutes and may be applied in on-off pulses. If pulsed acoustic energy is utilized a longer total application time may be required.

### BRIEF DESCRIPTION OF THE DRAWINGS

FIG. 1 is a schematic view of the testing fixture of the present invention in contact with bone cement;

FIG. 2 shows the temperature measurements for thermocouple #1 in test #1;

FIG. 3 shows the temperature measurements for test #2 for thermocouple #1 (channel 1), #2 (channel 2), and #3 (channel 3);

FIG. 4 shows the temperature measurements for test #3 for thermocouple #1 (channel 1), #2 (channel 2), and #3 (channel 3);

FIG. 5 is an illustration of the cured cement in the area of the horn tip in test #1;

FIG. 6 is an illustration of the cured cement in the area of the horn tip in test #2;

FIG. 7 is an illustration of the cured cement in the area of the horn tip in test #3; and

FIG. 8 shows the apparatus of the present invention in use with a prosthetic hip stem embedded in bone cement.

### DESCRIPTION OF THE INVENTION

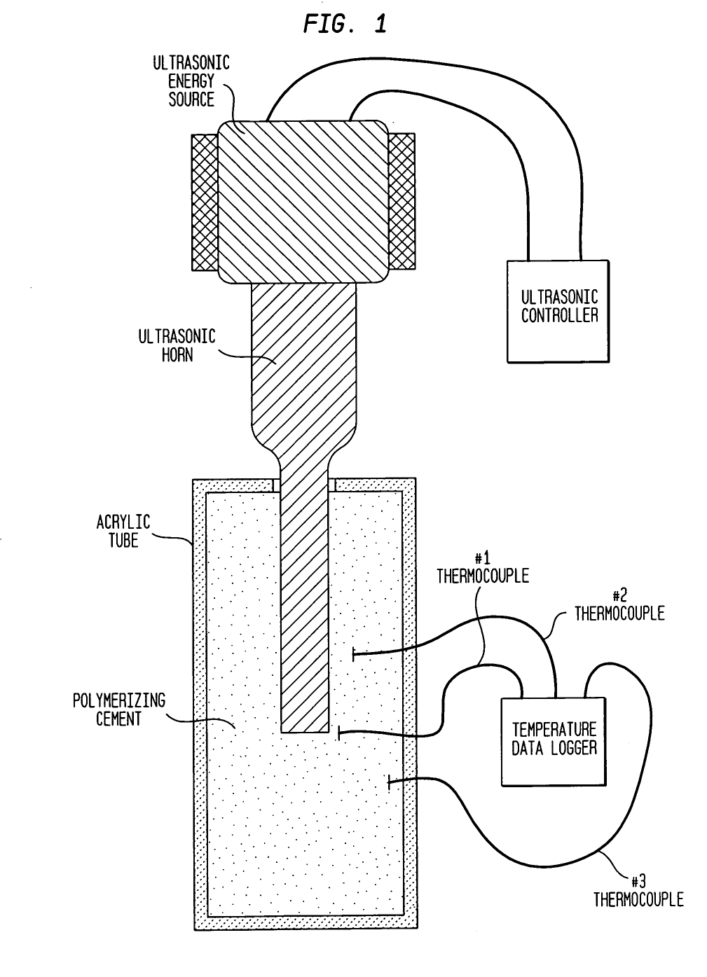

Three iterations of a setting time test were performed to evaluate the potential of using ultrasonics to accelerate the polymerization of Simplex® P acrylic bone cement using the test fixture of FIG. 1. All tests involved using a standard ½ inch diameter ultrasonic horn on a Sonicator 3000 (Misonix, Farmingdale, N.Y.) Ultrasonic Liquid Processor (energy source)to accelerate the polymerization of a single dose of Simplex® P cement that was hand mixed at atmospheric pressure and a room temperature of 23-24° C. The Simplex® P could also be mixed under vacuum using any standard mixing system.

Testing was performed using a ¾ inch ID acrylic tube designed to measure cement setting time at different locations within a polymerizing cement mantle. The fixture can utilize up to seven thermocouples to measure cement setting time at various user selected radial and axial locations. The testing fixture is shown schematically in FIG. 1 using three thermocouples.

For the first test, a single thermocouple was used, and for tests # 2 and # 3 three thermocouples were used, with the tip of the first thermocouple (thermocouple #1) positioned within a few millimeters of the tip of the ½ inch diameter ultrasonic horn. Temperature measurements were made at three locations for the second and third tests. In the second and third test thermocouple #1 was positioned within 2 millimeters of the tip of the ultrasonic horn. Thermocouple #2 was positioned approximately 12 mm proximal of the tip of the ultrasonic horn. This measurement was made in the middle of the 3 mm cement mantle that surrounded the ultrasonic horn. Thermocouple #3 was positioned approximately 10 mm distal of the tip of the ultrasonic horn, within a ½ millimeter of the inner wall of the acrylic tube.

Temperature measurements were made using 36-gage K-type thermocouple wires and recorded using a 4-channel data logger (Omega Series 3000). Time was started when the first drop of monomer came in contact with the prepolymerized powder. The cement was injected into the acrylic tubes from the bottom (in a retrograde manner) at a time of about four minutes after first contact for the first test and at a time of about 2 minutes after first contact for the second and third tests. (Table 1)

TABLE 1
Test Condition and Setting Times in [min:sec] for Tests 1, 2 and 3

| | Time of | | Ultrasonic | | Setting Time for | | |
|---|---|---|---|---|---|---|---|
| | Inserting | | Power | | Thermocouple | | |
| Test | Cement | Horn | On | Off | #1 | #2 | #3 |
| 1 | 4:02 | 4:52 | 5:34 | 6:16 | 6:11 | | |
| 2 | 2:02 | 2:55 | 3:03 | 4:06 | 3:52 | 4:19 | 6:21 |
| 3 | 2:02 | 2:50 | 2:56 | 4:44 | 4:29 | 6:32 | 7:05 |

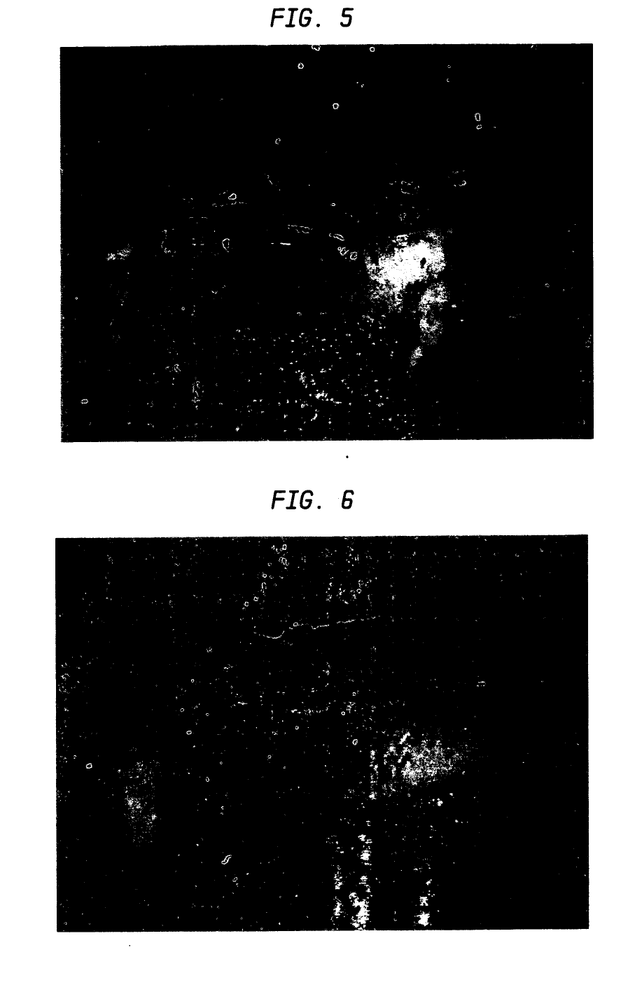

The ultrasonic horn was inserted about six inches into the cement approximately 1 minute after cement injection. Power was applied to the horn at 20 KHz frequency for the times and amplitude levels listed below in Table 2. After the cement was hardened in each test, the metallic horn was removed from the cement and the cement samples were sectioned longitudinally. Light photography was used to document the condition of the cement/horn interface. These results and photographs are shown in FIGS. 5-7.

TABLE 2
Power applied and amplitude levels for Tests 1, 2 and 3

| | Ultrasonic Power On | Ultrasonic Amplitude |
|---|---|---|
| Test # | (Min:Sec) | μm |
| 1 | 5:34 (334 sec) | 30 |
| 2 | 3:03 (183 sec) | 5 |
| 3 | 2:56 (176 sec) | 5 pulsed 3 sec on 5 sec off |

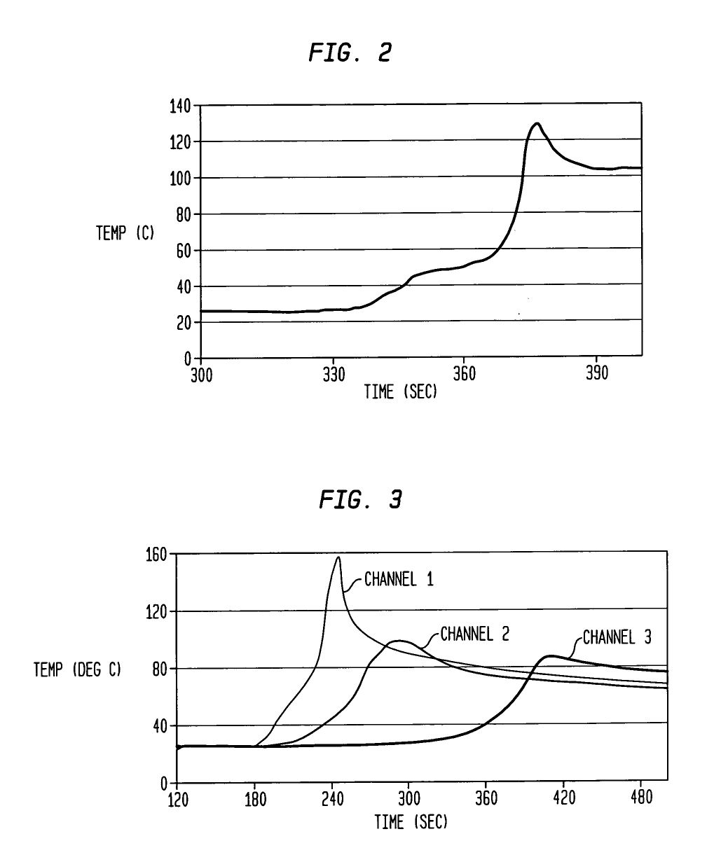

As shown in FIG. 2, the ultrasonic vibrations had an immediate effect on the cement closest to the tip of the horn. The thermocouple (#1) located within 2 mm of the horn tip measured an increase in temperature after the ultrasonic power was turned on. The temperature measured by this thermocouple continued to increase until the power was turned off, resulting in a temperature maximum at thermocouple #1 that corresponded with commencement of the ultrasonic vibrations.

Resultant setting times and maximum temperatures for each temperature measurement in each test are listed in tables 1 and 3. The cement nearest to the tip of the ultrasonic horn consistently reached a higher temperature maximum and consistently set faster than the cement at the other two locations. The cement that was proximal to the tip of the ultrasonic horn always set faster than the cement that was distal to the tip of the ultrasonic horn. All setting times measured under these test conditions are substantially decreased from the normal Simplex P setting time of 12 minutes measured in molds conforming to ASTM 451-99a, at a room temperature of 23 C.

The setting times for the cement nearest the tip of the horn (thermocouple #1) occurred very rapidly after applying ultrasonic energy to the cement. The setting times were 37 seconds, 46 seconds, and 1 minute 33 seconds after the initiation of ultrasonic vibrations for tests 1, 2, and 3 respectively.

TABLE 3
Maximum Temperature (Deg C.) for Tests 1, 2, and 3

| Test # | Temp#1 (Tip) | Temp#2 | Temp#3 |
|---|---|---|---|
| 1 | 131.1 | | |
| 2 | 160.3 | 99.5 | 88.8 |
| 3 | 115 | 87.3 | 87.2 |

The higher amplitude ultrasonic conditions for test #1 resulted in very rapid polymerization of the cement nearest the tip of the ultrasonic horn. An extensive network of voids was formed where the tip of the ultrasonic horn was in contact with the polymerizing cement. The cement was also more whitened in appearance near the tip of the horn in comparison to the cement next to the more proximal regions of the horn.

The conditions for test #2 utilized the lowest available ultrasonic amplitude setting, 0.5, and this was applied continuously to the cement. As shown in FIG. 3, when compared to test #1 (FIG. 2), this test condition resulted in a decrease in the rate of cement heating, 1.9° C. less (10%) in the first 15 seconds, and an 8 second (21%) delay in cement setting time. The size of voids formed at the tip of the horn for test #2 (FIG. 6) appeared to be smaller for test #1 (FIG. 5). There was also less whitening of the cement in the vicinity of the horn tip.

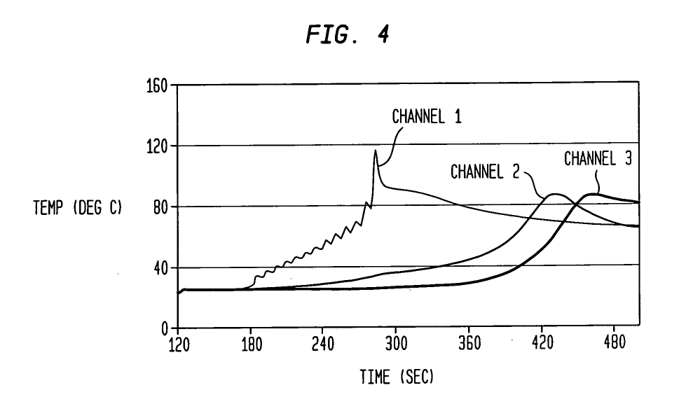

The conditions for test #3 utilized the lowest possible ultrasonic amplitude setting, 0.5, in combination with pulsed ultrasonics, 3 seconds on, and 5 seconds off. As shown in FIG. 4, this resulted in a discontinuous increase in cement temperature closest to the tip of the ultrasonic horn (Channel 1 in FIG. 4). The rate of temperature increase nearest to the tip of the horn was 10.3° C. (61%) less in the first fifteen seconds of heating in comparison to test #2 (FIG. 3). This resulted in a corresponding delay of 37 seconds, 2 minutes 13 seconds, and 44 seconds in cement setting times at thermocouple locations 1, 2, and 3 respectively.

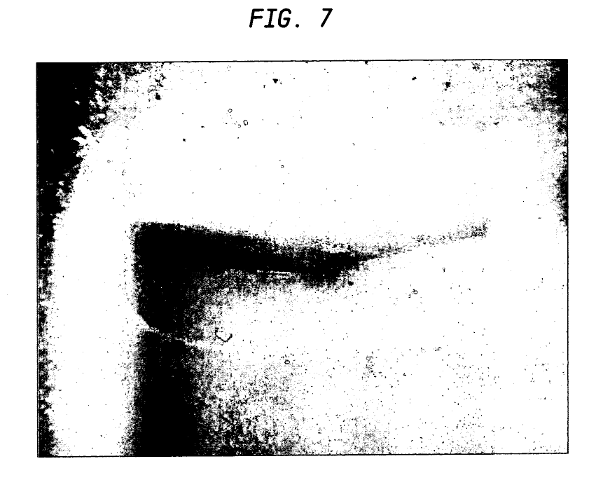

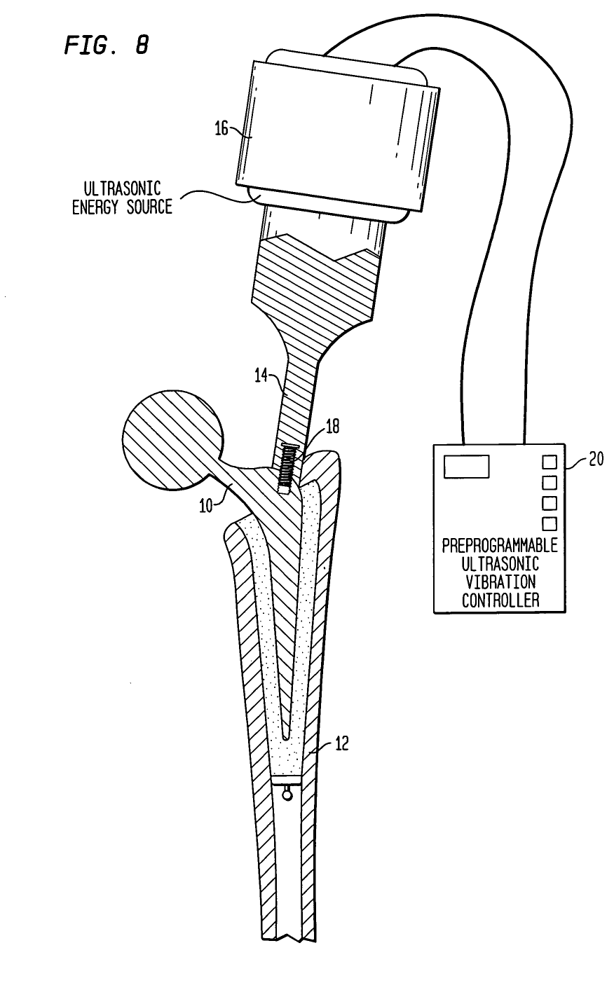

As shown in FIG. 7, there were fewer voids at the tip of the horn in test #3 in comparison to test #2 shown in FIG. 6. There was very little whitening of the cement in the vicinity of the horn tip. The cement at the tip of the horn was similar in appearance to the cement at more proximal locations next to the horn and within the mantle.

Under the conditions of this test, the addition of ultrasonic vibrations to a 3 mm mantle of cement resulted in a rapid increase in cement temperature and a corresponding decrease in cement setting time. When temperature was measured at more than one location within the polymerizing cement mantle, temperature maximum was highest and setting time was shortest for the measurements made closest to the tip of the ultrasonic horn.

FIG. 8 shows a preferred application of ultrasonic energy to a hip implant 10 located within a femur 12. Preferably the horn 14 of the ultrasonic energy source 16 is mechanically coupled to femoral component 10 by a threaded connection 18. A controller 20 is to control the energy input including amplitude and frequency.

Traditionally horns are designed, manufactured and tuned in order to vibrate efficiently in harmony with the ultrasonic energy source. In FIG. 8 the implant itself could be the horn of the ultrasonic sound generator. The invention allows utilization of a metallic implant as the horn for the ultrasonic sound generator. Rather than design implants that are tuned to a constant frequency ultrasonic energy source, a more sophisticated ultrasonic generator can be used to vibrate these complex geometry horns, such as cemented femoral hip stems or free tibial or femoral components at desired frequencies and amplitude.

A Master Sonic MSG-2000 was used to vibrate a femoral hip stem. This commercially available device allows mechanical vibration of large surface areas of complex geometry horns. The device works by allowing adjustment and modulation of the ultrasonic vibration to be in tune with the horn. The technology is termed multi-frequency, multimode modulated (MMM).

MMM (Modulated, Multimode, Multifrequency) ultrasonic generators utilize technology capable of stimulating wideband sonic and ultrasonic energy, ranging in frequency from infrasonic up to the MHz domain, that propagates through arbitrary shaped solid structures.

In conventional ultrasonics technology the transducers and connected elements are designed to satisfy precise resonant conditions. To achieve maximum efficiency, all oscillating elements must be tuned to operate at the same resonant frequency. In contrast MMM technology was developed to breakaway from this restrictive “tuned model” by using advanced Digital Signal Processing (DSP) techniques to implement an intelligent feedback loop that allows adaptation to most any un-tuned, changing, or evolving mechanical system. Instead of optimizing acoustic elements to accept a specific resonant frequency operation, MMM systems use the intelligent DSP to adapt to the un-tuned load. The system continuously analyzes system feedback and optimizes a complex shaped electrical driving signal customized to each specific oscillating structure.

To remain compatible with standard transducers the MMM generators use an adjustable primary resonant frequency as a central carrier frequency that efficiently drives standard transducers in a modulated mode. The MMM driving oscillations are not fixed or random, rather they follow a consistent and evolving pulse-repetitive pattern, where frequency, phase and amplitude are simultaneously modulated by the control system. The optimized modulations provide a highly efficient transfer of electrical to mechanical energy and prevent the creation of problematic stationary or standing waves as typically produced by traditional ultrasonic systems operating at a single frequency.

MMM systems offer a high level of control through regulation and programming of all vibration, frequency, and power parameters using either a handheld control panel or a Windows PC software interface. The system's fine control extends excellent repeatability and produces highly efficient active power that may range from below 100 W up to many kW. MMM technology can drive, with high efficiency, complex mechanical system up to a mass of several tons and consisting of arbitrary resonating elements.

Due to the flexible nature of the MMM technology, a wide range of new or improved applications are possible. For example applications requiring high temperatures represent a problem to conventional transducers that are extremely sensitive to heat. Since MMM systems are not restricted to specific tuned elements it is now possible to address high temperature applications through the use of extended acoustic wave-guides (e.g. 1 to 3 meters in length). An extended wave-guide puts the necessary physical distance between the heat sensitive transducer and the high temperature load. A long wave-guide also provides a convenient mounting point for cooling jackets that will draw away excessive heat and protect the transducer.

Such generators may be obtained from MP Interconsulting, Marais 36, 2400 Le Locle, Switzerland.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for accelerating the set time of acrylic bone cements comprising
mixing a monomer and a polymer of a two-part acrylic bone cement system;
placing the mixture within a bone prior to setting; and
thereafter applying acoustic energy from an ultrasonic sound generator to the unset mixture by applying acoustic energy to the acrylic cement mixture from the generator through a vibrating element.

2. The method as set forth in claim 1 wherein the frequency of the sound is 20 KHz.

3. The method as set forth in claim 1 wherein the acoustic energy is applied for less than two minutes.

4. The method as set forth in claim 1 wherein the amplitude of the acoustic energy varies from 5 to 30 μm.

5. The method as set forth in claim 1 wherein the acoustic energy is applied in pulses.

6. The method as set forth in claim 1 wherein the horn is attached to an implant embedded in the acrylic cement.

7. A method for accelerating the polymerization of acrylic bone cements comprising placing a horn of an ultrasonic acoustical generator in or adjacent a partially polymerized acrylic bone cement system and applying acoustic energy from the generator through the horn and into the partially polymerized bone cement system.

8. The method as set forth in claim 7 wherein the frequency of the acoustic energy is 20 KHz.

9. The method as set forth in claim 7 wherein the acoustic energy is applied for less than two minutes.

10. The method as set forth in claim 7 wherein the amplitude of the acoustic energy varies from 5 to 30 μm.

11. The method as set forth in claim 7 wherein the acoustic energy is applied in pulses.

12. The method as set forth in claim 7 wherein the horn is attached to an implant embedded in the acrylic cement.

13. The method as set forth in claim 7 wherein the horn is attached to an implant embedded in the acrylic cement.

14. A method for accelerating the cure time for an acrylic bone cement comprising applying ultrasonic energy to the cement during curing.

15. The method as set forth in claim 1 wherein the vibrating element is a horn coupled to the ultrasonic sound generator.

16. The method as set forth in claim 1 wherein the vibrating element is a metal implant for placement in the bone cement.

17. The method as set forth in claim 16 wherein the ultrasonic sound generator is a multi-frequency, multimode, modulated ultrasonic energy source.

18. The method as set forth in claim 1 wherein the ultrasonic sound generator is a multi-frequency, multimode, modulated ultrasonic energy source.
